(19) United States
(12) Patent Application Publication
(10) Pub. No.: US 2007/0045049 A1
(43) Pub. Date: Mar. 1, 2007

(54) Display and method of selling shutters

(76) Inventor: Keng Hao Nien, Changhua Hsien (TW)

Correspondence Address: BUCHANAN INGERSOLL & ROONEY PC, P.O. BOX 1404, ALEXANDRIA, VA, 22313-1404, US

(21) Appl. No.: 11/211,276

(22) Filed: Aug. 25, 2005

(51) Int. Cl. A61F 13/15; E04H 3/00

(52) U.S. Cl. 186/52

(57) ABSTRACT

In a display and method of selling shutters in which at least two sets of shutter panels, T-strips and spacers are displayed at a home center or other retail location. The homeowner measures the window opening and takes the measurement to the home center. The customer then selects at least two shutters from one of the sets of shutters on display and selects a T-strip from one of the sets of T-strips such that the selected shutters and T-strip can be assembled by the customer to fit the opening over which the customer desires to install the shutters. The components can be assembled in a manner so that any gap between adjacent shutters when those shutters are in a closed position is covered by the T-strip. When appropriate the customer may select spacers which are placed between each shutter panel and the window frame.

### FIELD OF THE INVENTION

The present invention relates to shutters of the type having a series of louvers in a rectangular frame and methods of selling such shutters.

### BACKGROUND OF THE INVENTION

Shutters which fold over window and door openings typically have a series of adjustable louvers in a rectangular frame and have been in use for a significant period of time. The cost and expertise required for installation varies greatly. Most homeowners do not have the tools or the skills to properly install many of the shutters on the market today. Therefore, user installations typically tend to be roughshod over-the-window-opening installations in which supports are simply bolted or screwed onto the walls in a room to both sides of a window opening. The spacing of the supports is generally dictated by the amount of space occupied by the shutter sections. As such, the supports typically are located spaced from the window opening and the result has a shoddy look and is mismatched to the opening. From within the room, the window covering protrudes out from the wall.

The “professional” installation generally requires stripping to be added to the inside of a window opening. The shutter support members are further attached, with continual measurements, marking, cutting, sanding and re-painting. Where the width distance needs to be reduced, and in order to keep the shutters balanced, an amount of material must be planed from the edges of the shutters in an even fashion. Each edge which is planed must be re-sanded and re-painted with time for drying and hopefully that no additional time will be required for re-fitting.

Where an installer does not measure exactly the distance to be cut, he will either have to repeat the exercise or scrap the materials, or perhaps leave a gap at the center. The center gap is a problem both in professional installations and for user installed configurations. The center light gap can be overly wide and even where it is kept to a minimum any orientation of the shutters other than parallel will produce a noticeable wedge shaped light projection. The elimination of the light gap should be done in a way which not only provides adequate covering, but which also looks natural and blends into the overall visual theme of the shutter system.

What is therefore needed is a shutter system which enables a user to perform a custom installation without the cutting, planing and painting which is involved in typical custom installations. What is further needed is a system which is forgiving and allows for fine adjustments. Such a shutter system should contain sets of standard components displayed in a single location which a homeowner can purchase shutters for most, if not all, standard size windows. The components should be easy to assemble and install using simple tools by most homeowners.

### SUMMARY OF THE INVENTION

I provide a display and method of selling shutters in which at least two sets of shutter panels, T-strips and spacers are displayed at a home center or other retail location. The homeowner measures the window opening and takes the measurement to the home center. The customer then selects at least two shutters from one of the sets of shutters on display and selects a T-strip from one of the sets of T-strips such that the selected shutters and T-strip can be assembled by the customer to fit the opening over which the customer desires to install the shutters. Moreover these components can be assembled in a manner so that any gap between adjacent shutters when those shutters are in a closed position is covered by the T-strip. When appropriate the customer may select spacers which are placed between each shutter panel and the window frame.

I prefer to also offer written installation instructions to customers at the retail location.

I further provide to offer knobs and hinges attached to the shutters. However, these items could be offered separately.

I further prefer to provide an adhesive or double-sided tape on the T-strip.

Other objects and advantages will become apparent from a description of certain present preferred embodiments thereof illustrated in the drawings.

### BRIEF DESCRIPTION OF THE DRAWINGS

FIG. 1 is a perspective view of a pair of shutters mounted on a window frame and in a closed position.

FIG. 2 is an exploded view of an upper portion of the shutters shown in FIG. 1.

FIG. 3 is a top plan view of the shutters shown in FIG. 2 in a partially open position.

FIG. 4 is a top plan view similar to FIG. 3 showing the shutters in a closed position.

FIG. 5 is a perspective view of a present preferred display of shutter components in accordance with the present invention.

### DESCRIPTION OF THE PREFERRED EMBODIMENTS

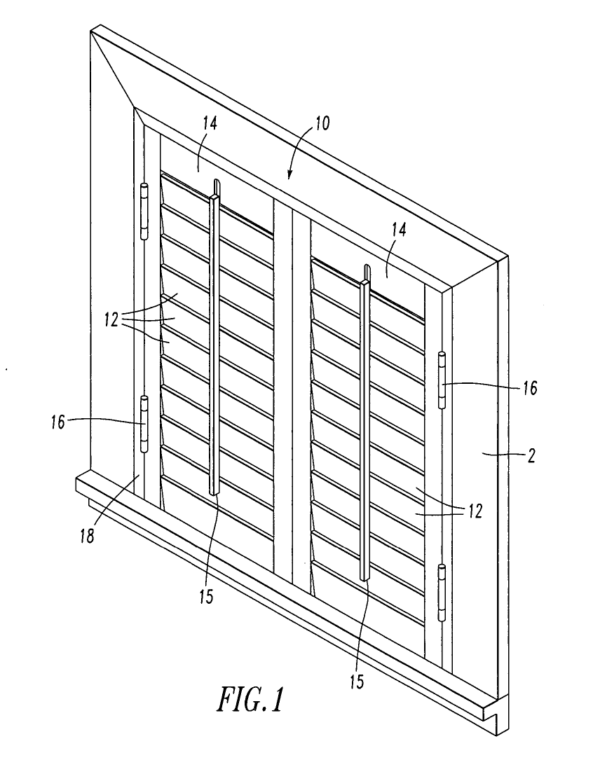

Shutters typically are installed in a window frame 2 in pairs as shown in FIG. 1. Each shutter 10 has a series of louvers 12 in a rectangular frame 14. Usually, a control rod 15 is attached to the center of each louver 12 and is used to open and close the louvers. Hinges 16 are provided along one vertical side of the shutter for attachment to the window frame 2. Sometimes a spacer strip or mounting strip 18 is provided between the shutter and the window frame.

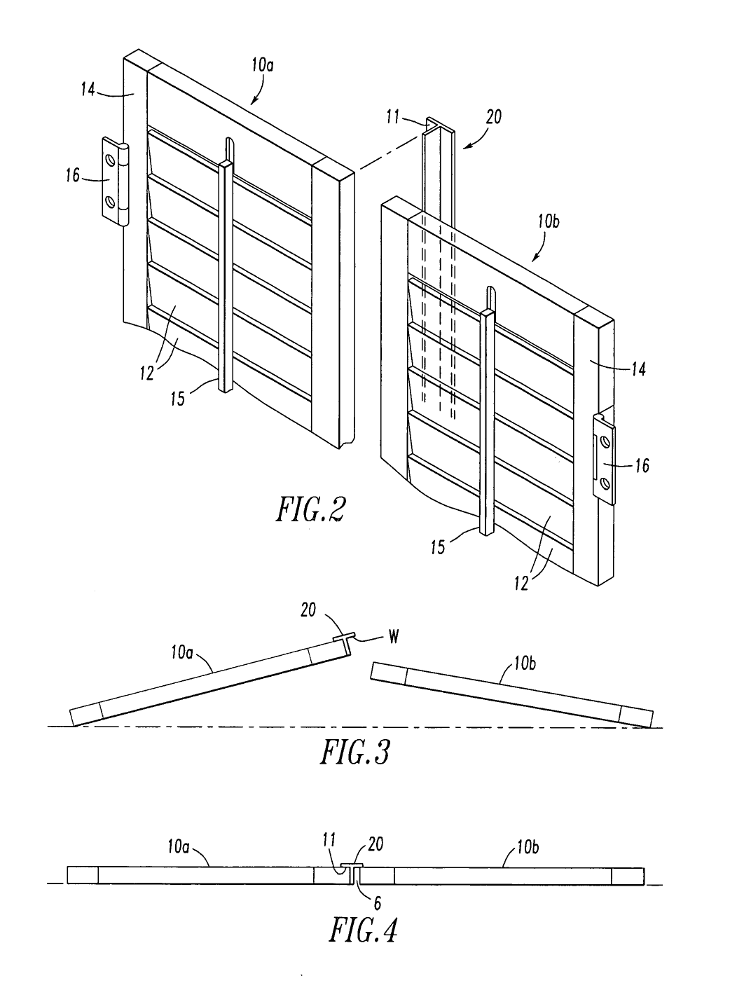

If the combined width of the shutters is less than the width of the window opening, there will be a gap between the shutters when the shutters are in a closed position. This will allow light to pass between the shutters and is usually undesirable. A common way to eliminate such a gap is to provide a strip along the edge of one of the shutters. This strip may be a flat piece, an L-shaped piece or a T-shaped piece. I prefer to use a T-shaped piece 20 such as shown in FIG. 2. As seen most clearly in FIGS. 3 and 4, the T-shaped piece 20 is attached to the edge of one shutter 10. When the shutter is closed the T-shaped piece will extend over the gap 6 between the shutters 10a and 10b. The T-shape of the strip makes it easy for the installer to attach the strip so that the strip is straight relative to the shutter. That is so, because two faces of the strip will abut adjacent surfaces of the shutter. The T-strip may be attached by nails, staples or an adhesive. I prefer to provide an adhesive covered by a release strip on the surface 11 of the T-shaped piece 20. That surface abuts the shutter 106 when the T-strip is installed. This adhesive could be double-sided tape. The adhesive enables the person assembling the shutters to easily attach the T-strip to one of the shutters. If desired a second adhesive, or strip of double-sided tape, could be attached to the adjacent side of the T-strip which is normal to the surface containing the first adhesive strip.

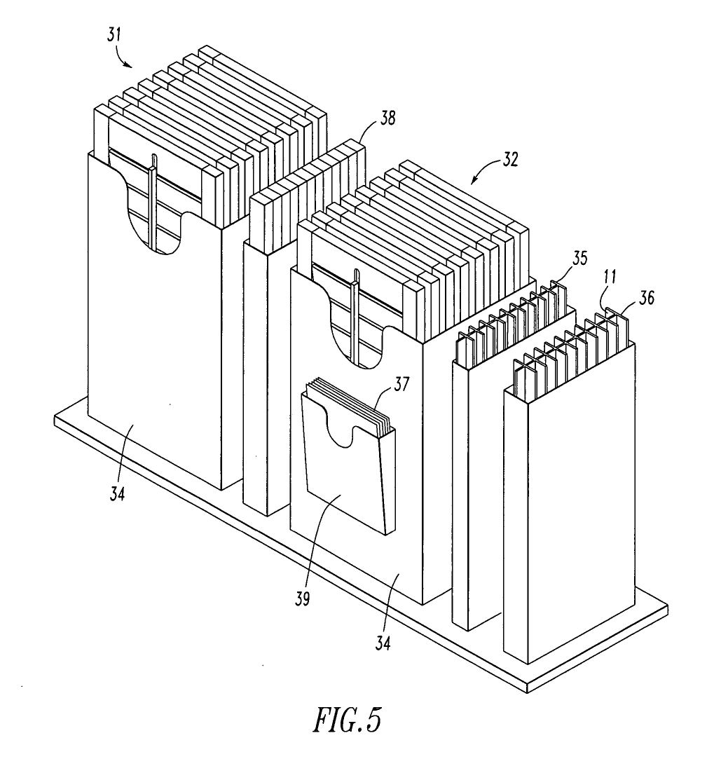

Referring to FIG. 5, I provide a display 30 containing at least two sets of shutters 31, 32. The shutters within each set of are the same width. But the shutters 31 in one set will have a different width from the shutters 32 in a second set. The shutters are preferably displayed in cut-down boxes 34 at a home center or other retail store location. Also provided are sets of T-shaped pieces or T-strips 35, 36. Each T-strip 35 or 36 in one set has the same width. However, the T-strips in one set will have a different width from the width of the T-strips in another set. In one preferred display I provide two sets of shutters one set having a width of 10 inches. I also provide two sets of T-strips one set having a width W of one inch from the center post to the outer edge and the second set having a width W of two inches from the center post to the outer edge. I may also provide at least one set of spacer strips or mounting strips 38 having a width between one-half inch and one inch. Hinges and mounting screws may be attached to each shutter or sold separately. A set of instructions 37 for assembling the displayed components can be contained in a rack 39 that is clipped onto one of the boxes containing the shutters. All of these components are preferably provided near one another in a defined display area. Indeed, they may all be contained in one rack or floor display.

A homeowner or other customer measures the width of the window opening over which he or she intends to place the shutters. The customer then goes to the display shown in FIG. 5 and selects a pair of shutters, a T-strip and possibly a pair of mounting strips that can be assembled to have a combined width that is not greater than the window opening and any gap between the shutters is covered by the T-strip when the shutters are closed.

The present method of selling shutters provides advantages for both homeowners and retailers. The home owner can immediately obtain a set of shutters for any common size window. The shutters are easy to install. The retailers can offer the shutters for all common window sizes by stocking standard size shutters, T-strips and mounting strips.

The present preferred shutters shown in the drawings are installed to have a single pair of shutters in the window opening. Some customers may prefer to use two or three pairs of shutters with one shutter from each pair being connected to a shutter from another pair by one or more hinges. The present system for selling shutters can easily accommodate these customers. Indeed, a retailer may choose to only sell shutters of smaller widths such as six or eight inches, with the expectation that multiple pairs of shutters would be purchased for each window.

The shutters, T-strips and mounting strips could be made of wood or vinyl. Indeed, many of the shutters currently in the marketplace could be sold using the present method.

Although I have described and illustrated certain present preferred embodiments of my display and method for selling shutters, the invention is not limited thereto and may be variously embodied within the scope of the following claims.

I claim:

1. A method of selling shutters of the type having a set of louvers in a rectangular frame, the frame having a height and width, comprising:
displaying at least two sets of shutters, each shutter within a set having a same width and the width of a shutter in one set being different from the width of a shutter in another set,
displaying at least two sets of T-strips each T-strip within a set having a same width and the width of a T-strip in one set being different from the width of a T-strip in another set, and
allowing a customer who has a measurement of an opening over which the customer desires to install a pair of shutters to:
select at least two shutters from one of the at least two sets of shutters and select a T-strip from one of the at least two sets of T-strips such that the selected shutters and T-strip can be assembled by the customer to fit the opening over which the customer desires to install the shutters in a manner so that any gap between adjacent shutters when those shutters are in a closed position is covered by the T-strip.

2. The method of claim 1 wherein each shutter is comprised of two panels connected together by at least one hinge, each panel having a set of louvers in a frame.

3. The method of claim 1 wherein the shutters are wood or vinyl.

4. The method of claim 1 also comprising providing to the customer written instructions for installing the shutters.

5. The method of claim 1 wherein each T-strip has an adhesive applied to at least one surface of the T-strip.

6. The method of claim 5 wherein the adhesive is double sided tape.

7. The method of claim 1 wherein the shutters in at least one of the sets of shutters is comprised of a pair of shutter panels connected together by at least one hinge.

8. A method of selling shutters of the type having a set of louvers in a rectangular frame, the frame having a height and width, comprising:
displaying at least two sets of shutters, each shutter within a set having a same width and the width of a shutter in one set being different from the width of a shutter in another set,
displaying at least two sets of T-strips each T-strip within a set having a same width and the width of a T-strip in one set being different from the width of a T-strip in another set,
displaying at least one set of mounting strips, each mounting strip within a set having a same width,
allowing a customer who has a measurement of an opening over which the customer desires to install a pair of shutters to:
select at least two shutters from one of the at least two sets of shutters, select a T-strip from one of the at least two sets of T-strips and select two mounting strips such that the selected shutters, T-strip and mounting strips can be assembled by the customer to fit the opening over which the customer desires to install the shutters in a manner so that any gap between adjacent shutters when those shutters are in a closed position is covered by the T-strip.

9. The method of claim 8 wherein each shutter is comprised of two panels connected together by at least one hinge, each panel having a set of louvers in a frame.

10. The method of claim 8 wherein the shutters are wood or vinyl.

11. The method of claim 8 also comprising providing to the customer written instructions for installing the shutters.

12. The method of claim 8 wherein each T-strip has an adhesive applied to at least one surface of the T-strip.

13. The method of claim 12 wherein the adhesive is double sided tape.

14. The method of claim 1 wherein the shutters in at least one of the sets of shutters is comprised of a pair of shutter panels connected together by at least one hinge.

15. A display for selling shutters comprising:
at least two sets of shutters, each shutter within a set having a same width and the width of a shutter in one set being different from the width of a shutter in another set, each set of shutters being displayed separate from all other sets of shutters, and
at least two sets of T-strips each T-strip within a set having a same width and the width of a T-strip in one set being different from the width of a T-strip in another set, each set of T-strips being displayed separate from all other sets of T-strips,
the sets of shutters and sets of T-strips being located proximate to one another and within a defined display area.

16. The display of claim 15 also comprising at least one set of mounting strips within the defined display area.

17. The display of claim 15 wherein each shutter is comprised of two panels connected together by at least one hinge, each panel having a set of louvers in a frame.

18. The display of claim 15 wherein the shutters are wood or vinyl.

19. The display of claim 15 also comprising at least one set of written instructions for installing the shutters within the defined display area.

20. The display of claim 15 wherein each T-strip has an adhesive applied to at least one surface of the T-strip.

21. The display of claim 20 wherein the adhesive is double sided tape.

22. The display of claim 15 wherein the shutters in at least one of the sets of shutters is comprised of a pair of shutter panels connected together by at least one hinge.
